(19) United States
(12) Patent Application Publication
(10) Pub. No.: US 2006/0111717 A1
(43) Pub. Date: May 25, 2006

(54) Implant with a threaded bore for a bone screw

(75) Inventors: Thomas Saueressig, Tuttlingen (DE); Hans-Werner Stedtfeld, Nuernberg (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/260,670

(22) Filed: Oct. 26, 2005

Related U.S. Application Data
(63) Continuation of application No. PCT/EP04/04710, filed on May 4, 2004.

(30) Foreign Application Priority Data
May 9, 2003 (DE) 103 20 855.0

(51) Int. Cl. A61B 17/56

(52) U.S. Cl. 606/64

(57) ABSTRACT

In order to secure the screw-in position of a bone screw in an optimum manner in the case of an implant with a threaded bore for receiving the bone screw screwed into it, it is suggested that a fixing member be held on the implant in the direction of the bore adjacent to the threaded bore, this fixing member having an opening which is aligned with the bore and has a diameter which is smaller than the external diameter of the outer edge of the threads of the bone screw, and that the fixing member consist of a material which is so soft that the threads of the bone screw dig into this material or displace it when the bone screw is screwed into the threaded bore of the implant.

This application is a continuation of International application number PCT/EP2004/004710 filed on May 4, 2004.

The present disclosure relates to the subject matter disclosed in International application number PCT/EP2004/004710 of May 4, 2004 and German application number 103 20 855.0 of May 9, 2003, which are incorporated herein by reference in their entirety and for all purposes.

### BACKGROUND OF THE INVENTION

The invention relates to an implant with a threaded bore for receiving a bone screw screwed into it.

Implants are often secured to bones by means of bone screws which themselves have a thread for receiving the bone screw. The bone screw is then screwed not only into the implant but also into the bone. This may be necessary, for example, to fix bone fragments in place; in this way, it is intended for diverse bone fragments to be joined securely to one another via the implant for the purpose of healing. Implants of this type may be designed as bone plates or also as intramedullary nails which are inserted into the medullary space and are screwed to the bone fragments through the wall of the surrounding tubular bone.

It has been found that the bone screws can loosen during the healing process. This happens, in particular, during the rehabilitation phase due to movements made by a patient. In this respect, the bone screws can tilt slightly in the threaded bore since a certain amount of play is always present between the bone screw and the threaded bore. A slight rotational movement is also possible and this can lead to the bone screw being screwed out.

The object of the invention is to design a generic implant such that any unintentional loosening of this type or even screwing out of the bone screw is prevented.

### SUMMARY OF THE INVENTION

This object is accomplished in accordance with the invention, for an implant of the type described at the outset, in that a fixing member is held on the implant in the direction of the bore adjacent to the threaded bore, this fixing member having an opening which is aligned with the bore and has a diameter which is smaller than the external diameter of the threads of the bone screw, and that the fixing member consists of a material which is so soft that the threads of the bone screw dig into this material or displace it when the bone screw is screwed into the threaded bore of the implant.

With this fixing member, a screw brake is produced, so to speak, in which the screwed-in bone screw is held in a pressing fit. As a result, any tilting of the bone screw in the threaded bore is prevented and this also leads to the screwing out of the bone screw during body movements being avoided. The threads of the bone screw deform the material of the fixing member and bring about the desired secure hold in the threaded bore in this way. In this respect, it is of significance that the bone screw is guided reliably during the screw-in process itself by way of the screwing into the threaded bore and the deformation of the fixing member can therefore be carried out in an exactly defined manner. There is, therefore, no risk of the bone screw being deflected out of the desired direction by the fixing member during its insertion since this desired direction is clearly predetermined by the threaded bore of the implant.

The fixing member preferably displays elastomeric properties and so it can make way for the bone screw elastically and then abut elastically on it.

It may, consist, for example, of a plastic material.

In a first, preferred embodiment it is provided for the fixing member to be designed as a ring arranged concentrically to the threaded bore.

A space can exist between the ring and the threaded bore so that the bone screw can be screwed into the threaded bore first of all without any contact to the fixing member and comes into contact with the fixing member only during further screwing in.

It is favorable when the ring is inserted into an inner circumferential groove of the threaded bore; threads for the bone screw are then located on both sides of the ring when seen in the screw-in direction. It is, however, also possible for the ring to be inserted into an inner circumferential groove of an extension of the threaded bore; threads for the bone screw are then provided in the implant only on one side of the ring, namely in such a manner that the bone screw is screwed first of all into the threads of the threaded bore and then into the fixing member.

It is favorable when the ring is slotted; this facilitates its insertion into the inner circumferential groove.

In another, preferred embodiment it is provided for the threaded bore to be arranged in the wall of a tubular implant and the fixing member to be designed as a plug inserted into the interior of the tubular implant. Such a design is favorable, in particular, when the implant has the shape of an intramedullary nail.

Several openings which are aligned with threaded bores for bone screws of the implant may be provided in the fixing member so that several bone screws can be secured in their threaded bores with one fixing member.

The following description of preferred embodiments of the invention serves to explain the invention in greater detail in conjunction with the drawings.

### BRIEF DESCRIPTION OF THE DRAWINGS

FIG. 1: shows a side view of an intramedullary nail with a bone screw turned into it and a screw brake in the form of a plug inserted into the intramedullary nail;

FIG. 2: shows a sectional view along line 2-2 in FIG. 1 and

FIG. 3: shows a partial sectional view similar to FIG. 2 in a modified embodiment with a screw brake in the form of a ring inserted into the implant.

### DETAILED DESCRIPTION OF THE INVENTION

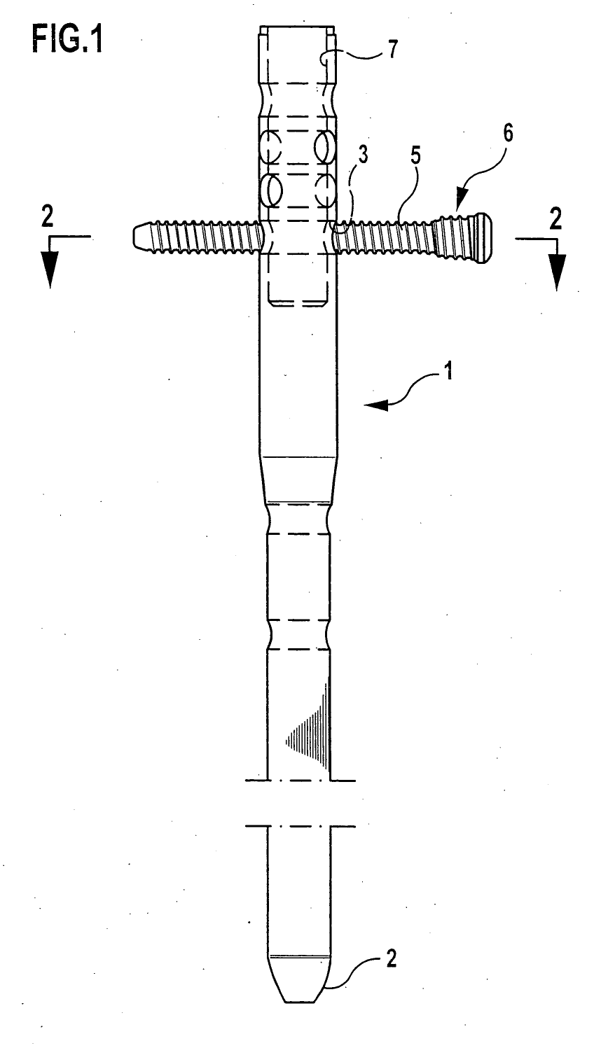

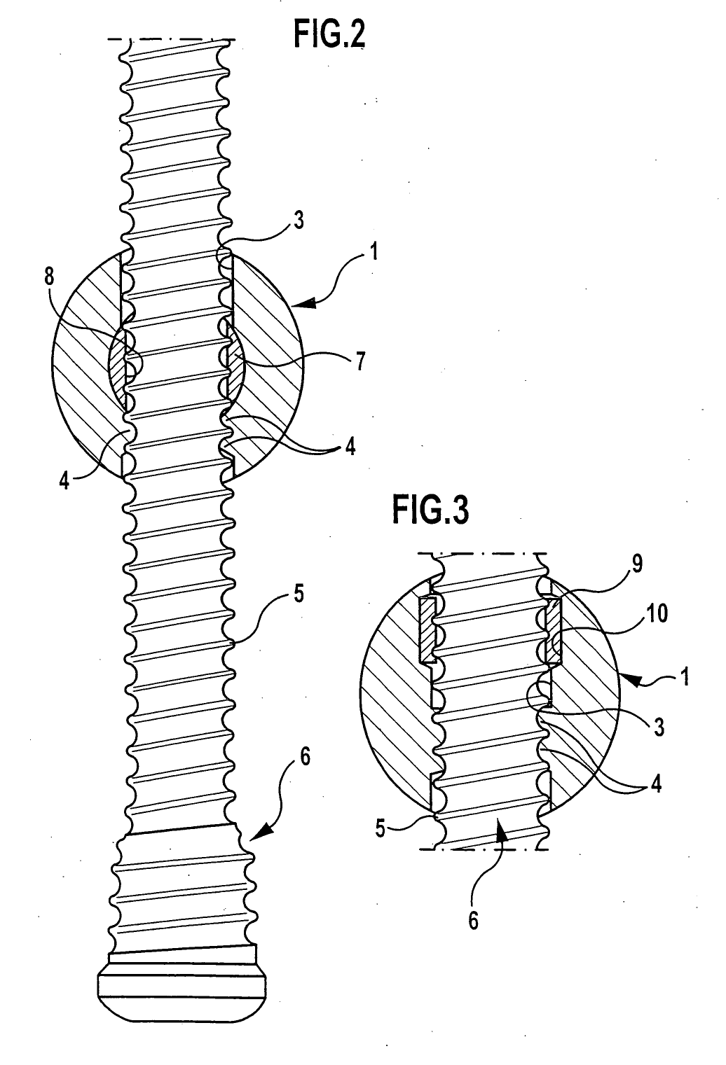

An intramedullary nail 1 is illustrated in FIGS. 1 and 2 and this is essentially of a tubular design and can be introduced into the medullary space of a tubular bone with a blunt point 2 in order to fix wall fragments of this tubular bone via the intramedullary nail 1. Several diametrical bores 3 are provided in the tubular shaft 3 of the intramedullary nail 1 and these have threads 4 in the wall of the intramedullary nail 1 on one side of the intramedullary nail 1 for receiving the threads 5 of a bone screw 6 whereas threads of this type are not present on the oppositely located wall side of the intramedullary nail 1; in this case, the bore 3 has a diameter which is somewhat greater than the external diameter of the bone screw 6 so that this can be pushed through this part of the bore 3 without any screwing in.

A plastic plug 7 consisting of an elastomeric material is pushed into the interior 7 of the intramedullary nail 1 from the upper side and this plug has a bore 8 aligned with the bore 3 in the area thereof. The internal diameter of this bore 8 is smaller than the external diameter of the threads 5 of the bone screw 6 and so these threads 5 dig into the material of the plastic plug 7 when the bone screw 6 is screwed in and, in this respect, displace this material. The plastic plug 7 preferably consists of an elastic material so that the material again abuts elastically on the threads 5 of the bone screw 6 once the bone screw 6 has been screwed in and, as a result, the bone screw 6 is secured in the bore 3. As a result, the play of the bone screw 6 in the bore 3 is prevented; any unintentional screwing of the bone screw out of the threads 4 of the bore 3 is also prevented.

Several bores 3 are arranged one above the other in the intramedullary nail 1 and each of these bores has a corresponding bore in the plastic plug 7 associated with it so that bone screws can be screwed into all the bores 3 and can then be fixed in their screwed-in position by the same plastic plug 7.

Whereas, in the embodiment of FIGS. 1 and 2, the bone screws 6 are secured in the screwed-in state by a screw brake in the form of a plastic plug, a plastic ring 9 is used as screw brake in the embodiment of FIG. 3 and this is inserted into an inner circumferential groove 10 in the bore 3; for this purpose, the plastic ring 9 is of a slotted design.

The embodiment illustrated in FIG. 3 is particularly suitable for a solid intramedullary nail which does not, therefore, have any tubular interior space in the area of the bore 3. In the embodiment illustrated, a space is provided between the threads 4 of the bore 3 and the circumferential groove 10 which accommodates the plastic ring 9; therebetween, the bore 3 encloses the bone screw 6 in spaced relationship without engaging in the threads 5 of the bone screw 6.

In this embodiment, as well, the plastic ring 9 preferably consists of an elastomeric material which abuts on the screwed-in bone screw 6 and secures the threads 5 and, with them, the bone screw 6 against any tilting or turning.

1. Implant with a threaded bore for receiving a bone screw screwed into it with a fixing member held on the implant in the direction of the bore adjacent to the threaded bore, said fixing member having an opening aligned with the threaded bore and having a diameter smaller than the external diameter of the outer edge of the threads of the bone screw, wherein the fixing member consists of a material so soft that the threads of the bone screw dig into this material or displace it when the bone screw is screwed into the threaded bore of the implant, wherein the threaded bore is arranged in the wall of a tubular implant and the fixing member is designed as a plug inserted into the interior space of the tubular implant.

2. Implant as defined in claim 1, wherein the fixing member displays elastomeric properties.

3. Implant as defined in claim 1, wherein the fixing member consists of a plastic material.

4. Implant as defined in claim 2, wherein the fixing member consists of a plastic material.

5. Implant as defined in claim 1, wherein several openings aligned with threaded bores for bones screws of the implant are provided in the fixing member.

6. Implant as defined in claim 2, wherein several openings aligned with threaded bores for bones screws of the implant are provided in the fixing member.

7. Implant as defined in claim 3, wherein several openings aligned with threaded bores for bones screws of the implant are provided in the fixing member.
